(19) United States
(12) Patent Application Publication
(10) Pub. No.: US 2022/0367044 A1
(43) Pub. Date: Nov. 17, 2022

(54) ELECTROCARDIOGRAM ANALYSIS MATCHING SUPPORT SERVICE SYSTEM

(71) Applicant: Wellysis Corp., Seoul (KR)

(72) Inventors: Young JUHN, Seoul (KR); Rick Hongryul KIM, Seoul (KR); Jong Woo KIM, Seoul (KR); Jung Soo KIM, Seoul (KR)

(73) Assignee: Wellysis Corp., Seoul (KR)

(21) Appl. No.: 17/357,124

(22) Filed: Jun. 24, 2021

(30) Foreign Application Priority Data
May 17, 2021 (KR) 10-2021-0063581

(51) Int. Cl. G16H 40/67; G16H 40/20; G16H 50/20; G16H 10/65; A61B 5/339; A61B 5/00; A61B 5/361; A61B 5/363; A61B 5/366

(52) U.S. Cl. CPC G16H 40/67; G16H 40/20; G16H 50/20; G16H 10/65; A61B 5/339; A61B 5/0006; A61B 5/361; A61B 5/363; A61B 5/366

(57) ABSTRACT

The present invention relates to an electrocardiogram analysis matching support service system that supports timely and real-time analysis of an individual's electrocardiogram, and it is characterized in that it includes:
a patient service app module that is installed and executed in a patient's mobile communication terminal, transmits an electrocardiogram measurement data received from a wearable electrocardiogram measurement device, requests for reading, and receives and displays the result of the reading; and
a patient and medical staff matching server that reads the electrocardiogram measurement data transmitted from the patient service app module using a deep learning trained artificial intelligence network model, selects pre-registered medical staff according to the result of the reading, and supports reading the electrocardiogram measurement data.

### TECHNICAL FIELD

The present invention relates to a telemedicine relay service system, and more particularly, to an electrocardiogram analysis matching support service system that supports timely real-time analysis (interpretation) of an individual electrocardiogram.

### BACKGROUND ART OF THE INVENTION

The active current generated by the myocardium according to the heartbeat can be induced to two suitable places on the body surface and recorded with an ammeter, and the recording of the myocardial activity current obtained in this way is called an electrocardiogram.

Electrocardiography is performed in patients with symptoms such as chest pain, dyspnea, or other diseases affecting the heart, such as high blood pressure, and it can be performed as a basic test to confirm heart function before general anesthesia in patients before surgery.

Electrocardiography is usually performed by an electrocardiogram measuring device (or measuring system), and such an electrocardiogram measuring device can be classified into various types of measuring devices depending on the use or purpose.

In general, when a patient feels heart-related symptoms (palpitation, dyspnea, and the like), they make an appointment for a hospital and receive a prescription for electrocardiography through consultation with a doctor. In some cases, a wearable electrocardiogram measuring device may be used for daily electrocardiography. In this case, after returning the device, the patient revisits the hospital to hear the diagnosis result.

As mentioned, in order to receive electrocardiography and diagnosis, time is unnecessarily delayed because a series of steps such as a hospital appointment, a hospital visit, an examination, a notification of the diagnosis result, and the like has to be gone through, and furthermore, from the patient's point of view, there is a limit of having to take the trouble of making multiple appointments and visiting the hospital and having to select the medical staff belonging to that hospital, and for the medical staff, they have no choice but to devote a lot of time to schedule management, device management, and billing management.

In particular, since it is a method of providing health-related data obtained from individual patients to medical institutions, there is a burden of paying a separate fee for re-diagnosis or obtaining the health-related data once provided to previous institutions and submitting thereof when visiting other medical institutions.

In addition, when a patient feels abnormal symptoms related to the heart, it is ideal to measure electrocardiogram data in real-time and receive a quick diagnosis in real-time, but since the current medical system does not support this, it causes a waste of time for a diagnosis of a heart disease.

### PRIOR ART LITERATURE

### Patent Literature

Patent Literature 1: Korean Registered Patent Publication No. 10-1791318
Patent Literature 2: Korean Registered Patent Publication No. 10-1598918

### SUMMARY OF INVENTION

### Technical Problems

Accordingly, the present invention is an invention devised to solve the above-described problems and inconveniences, and the main object of the present invention is to provide an electrocardiogram analysis matching support service system that can provide timely real-time analysis (interpretation) of an individual electrocardiogram, and furthermore, another object of the present invention is to provide an electrocardiogram analysis matching support service system capable of receiving an electrocardiogram diagnosis by freely selecting a doctor, regardless of distance and time, and

to provide an electrocardiogram analysis matching support service system that can match the best medical staff by applying different medical staff selection criteria according to the reading mode selected by a patient. In addition, another object of the present invention is to provide an electrocardiogram analysis matching support service system capable of minimizing payment of medical expenses by selectively providing one's own electrocardiogram measurement data to medical staff whenever necessary, and furthermore, the present invention can support the patient's own self-management by accumulating and managing the patient's own electrocardiogram measurement data, and it is to provide an electrocardiogram analysis matching support service system that can provide economic benefits to both patients and medical staffs compared to the current medical system.

### Technical Solution

An electrocardiogram analysis matching support service system according to an embodiment of the present invention for achieving the above object is characterized by comprising:

a patient service app module, installed and executed on a mobile communication terminal of a patient, that transmits an electrocardiogram measurement data received from a wearable electrocardiogram measurement device to a patient and medical staff matching server to request for a reading, and receives and displays the result of the reading; and a patient and medical staff matching server that reads the electrocardiogram measurement data transmitted from the patient service app module using a deep learning trained artificial intelligence network model, selects a pre-registered medical staff according to the result of the reading, and supports to read the electrocardiogram measurement data.

In the electrocardiogram analysis matching support service system including the above configuration, the patient service app module is characterized by selectively storing and transmitting the patient's electrocardiogram measurement data acquired for the previous n seconds and the electrocardiogram measurement data of the patient acquired for the next m seconds thereafter based on the input time when selectively inputting the symptom expression measurement mode by the patient. (The above n and m are natural numbers.)

Further, in the electrocardiogram analysis matching support service system including the above-described configuration, the patient service app module is characterized in that the current location information of a patient obtainable from the mobile communication terminal of the patient is transmitted together with information on the request for electrocardiogram reading, and

the patient and medical staff matching server is characterized by including a medical staff selection unit that reads patient's electrocardiogram measurement data using a deep learning trained artificial intelligence network model, selects some of the registered medical staff according to the result of the reading, and requests for electrocardiogram reading.

An electrocardiogram analysis matching support service system according to another embodiment of the present invention is characterized in that it includes:

a patient service app module, installed in the mobile communication terminal of a patient, that receives the electrocardiogram measurement data acquired from a wearable electrocardiogram measuring device and sends it to the patient and medical staff matching server along with information on the request for electrocardiogram reading, and receives and displays information on a list of medical staff responding to a request for electrocardiogram reading, and transmitting the medical staff information selected by the patient as feedback; and

a patient and medical staff matching server that transmits the request for electrocardiogram reading transmitted through the patient service app module to the registered medical staff communication terminal, and transmits the result of the reading obtained by providing the patient's electrocardiogram measurement data to the communication terminal of the medical staff responding to the request for electrocardiogram reading to the patient service app module.

In the electrocardiogram analysis matching support service system of this configuration, the patient service app module is characterized by comprising:

a UI control unit, which is configured to include an operation mode selection button to select a continuous measurement mode and a symptom expression measurement mode, a reading mode selection button to select a real-time reading and a full reading, and displaying medical staff list information; a data storage control unit that divides the received patient's electrocardiogram measurement data into date, measurement time, and operation mode and stores it in a patient's mobile communication terminal storage unit; and

a data transmission control unit for receiving or transmitting control of the patient's electrocardiogram measurement data between a wearable electrocardiogram device and the patient and medical staff matching server according to the selected operation mode or reading mode, wherein another feature is that the data storage control unit selects and stores the patient's electrocardiogram measurement data acquired for the previous n seconds and the patient's electrocardiogram measurement data acquired for the next m seconds based on the input time when the symptom expression measurement mode selection input is inputted. (The above n and m are natural numbers.)

Furthermore, the patient service app module is characterized by transmitting the current location information obtainable from a patient's mobile communication terminal together with the information of the request for electrocardiogram reading so as to be used for medical staff selection. Meanwhile, the patient and medical staff matching server is characterized by reading the patient's electrocardiogram measurement data using a deep learning trained artificial intelligence network model, and including a medical team selection unit that selects some of the registered medical staff according to the result of the reading and transmits the request for an electrocardiogram reading, and

another feature is that the medical staff selection unit selects the registered medical staff according to the degree of the ratio of VT and SVT to QRS, and rhythm detection in the patient's electrocardiogram measurement data, wherein medical staff is selected as the top priority selection criteria among the distance between a patient and a medical staff according to the above ratio, a medical staff who can read in real-time, and medical staff who responded first.

### Advantageous Effects of Invention

According to the above-described technical problem solving means, the electrocardiogram analysis matching support service system according to an embodiment of the present invention supports to receive an electrocardiogram diagnosis by matching a patient and a medical staff online regardless of distance and time, and thereby the patient has the advantage of being able to receive a timely, real-time remote diagnosis on the electrocardiogram measurement data obtained immediately before and immediately after the symptom expression time.

In addition, since the electrocardiogram analysis matching support service system according to an embodiment of the present invention automatically reads the patient's electrocardiogram measurement data, selects the best medical staff according to the result of the reading, and matches the patient with the medical staff, it has the advantage of being able to provide the best telemedicine service even in an environment that requires promptness. In addition, the present invention supports the selection and application of various medical staff selection criteria according to the patient's propensity, thereby not only capable of matching the medical staff suitable for the patient, but also capable of providing sales results extended through offline treatment based on the trust accumulated online.

### BRIEF DESCRIPTION OF DRAWINGS

FIG. 1 is an exemplary diagram of a peripheral configuration of an electrocardiogram analysis matching support service system according to an embodiment of the present invention.

FIG. 2 is an exemplary block configuration diagram of a patient service app module installed and executed in a patient's mobile communication terminal according to an embodiment of the present invention.

FIG. 3 is an exemplary configuration diagram of a patient and medical staff matching server according to an embodiment of the present invention.

FIG. 4 is an exemplary view of an electrocardiogram waveform according to an embodiment of the present invention.

FIG. 5 is an exemplary operation flow diagram of a patient service app according to an embodiment of the present invention.

FIG. 6 is an exemplary operation flow diagram of a patient and medical staff matching server 300 according to an embodiment of the present invention.

### DETAILED DESCRIPTION OF EMBODIMENTS

For the detailed description of the present invention, which will be described later, reference is made to the accompanying drawings, which illustrate specific embodiments in which the present invention may be practiced in order to clarify the objectives, technical solutions and advantages of the present invention. These embodiments are described in detail sufficient to enable a person skilled in the art to practice the present invention.

In addition, throughout the description and claims of the present invention, the term ‘comprises’ and variations thereof are not intended to exclude other technical features, additions, components or steps. Other objectives, advantages, and features of the present invention will be revealed to a person skilled in the art, in part, from this description, and in part from the practice of the present invention. The examples and drawings below are provided by way of example and are not intended to limit the invention. Furthermore, the present invention covers all possible combinations of the embodiments indicated herein. It is to be understood that the various embodiments of the present invention are different from each other, but need not be mutually exclusive. In addition, it is to be understood that the location or arrangement of individual components within each disclosed embodiment may be changed without departing from the spirit and scope of the present invention. Accordingly, the detailed description to be described below is not intended to be taken in a limiting sense, and the scope of the present invention, if properly described, is limited only by the appended claims, along with all scopes equivalent to those claimed by the claims.

Unless otherwise indicated or clearly contradicted by context in this specification, items referred to as the singular, unless otherwise required by the context, encompass the plural. In addition, in describing the present invention, a detailed description thereof will be omitted when it is determined that a detailed description of a related known configuration or function may obscure the subject matter of the present invention.

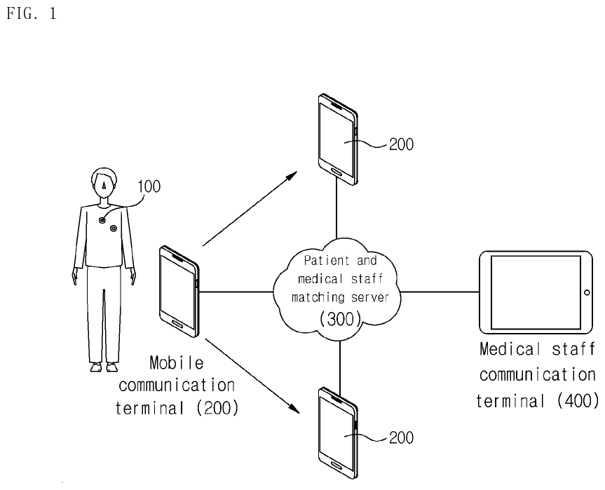

FIG. 1 is an illustration of a peripheral configuration diagram of an electrocardiogram analysis matching support service system according to an embodiment of the present invention, and the electrocardiogram analysis matching support service system according to an embodiment of the present invention comprises:

a patient service app module installed and executed in a patient's mobile communication terminal 200, in which the electrocardiogram measurement data wirelessly received from a wearable electrocardiogram measurement device 100 is transmitted to a patient medical team matching server 300 to request for a reading, and displays the result of the reading received from the patient medical team matching server 300; and

a patient and medical staff matching server 300 that reads the electrocardiogram measurement data transmitted from the patient service app module using a deep learning trained artificial intelligence network model, and selects pre-registered medical staff according to the result of the reading to support reading the electrocardiogram measurement data.

For reference, the patient service app module may be implemented as a set of code data that is installed in the memory of a patient's mobile communication terminal 200 and constitutes an executable application program. Since this patient service app module is installed and executed in the mobile communication terminal 200, the same reference number 200 of the mobile communication terminal is used.

An electrocardiogram analysis matching support service system according to another embodiment of the present invention that can be modified may include:

a patient service app module 200 installed in the patient's mobile communication terminal that receives the electrocardiogram measurement data obtained from a wearable electrocardiogram measurement device 100 and transmits it to a patient and medical staff matching server 300 together with information of a request for electrocardiogram reading, and receives and displays a list of medical staff information responding to the request for electrocardiogram reading, and transmits information of the medical staff selected by the patient as feedback.

It may include a patient and medical staff matching server 300 that transmits a request for electrocardiogram reading transmitted through the patient service app module 200 to a registered medical staff communication terminal 400, and transmits the result of the reading being obtained by providing the patient's electrocardiogram measurement data to the medical staff communication terminal 400 which responded to the request for electrocardiogram reading to the patient service app module 200.

In FIG. 1, a wearable electrocardiogram measurement device 100 includes: a plurality of electrocardiogram electrodes attached to the patient's body as a patch type to detect an electrocardiogram waveform; a signal preprocessing unit that removes noise from the electrocardiogram waveform obtained from the electrocardiogram electrodes, amplifies signals, and converts them into digital electrocardiogram measurement data; a communication unit for transmitting the electrocardiogram measurement data to the patient's mobile communication terminal 100 through wireless (BLE) communication; and a memory. The wearable electrocardiogram measurement device 100 having such a configuration may transmit an electrocardiogram measurement data to a mobile communication terminal in real-time according to the operation mode, or store the measured data for a certain period in a memory and then transmit it to the mobile communication terminal.

The medical staff communication terminal 400 is a computer system capable of connecting with a patient and medical staff matching server 300 in a wired or wireless way, in which a medical staff service application program that responds to a request for electrocardiogram reading and includes the necessary tools to read the electrocardiogram measurement data and enter the result of the reading is installed.

Hereinafter, the configuration of a patient's mobile communication terminal and a patient and medical staff matching server 300 in which a patient service app module 200 illustrated in FIG. 1 is installed will be described in more detail with reference to the accompanying drawings.

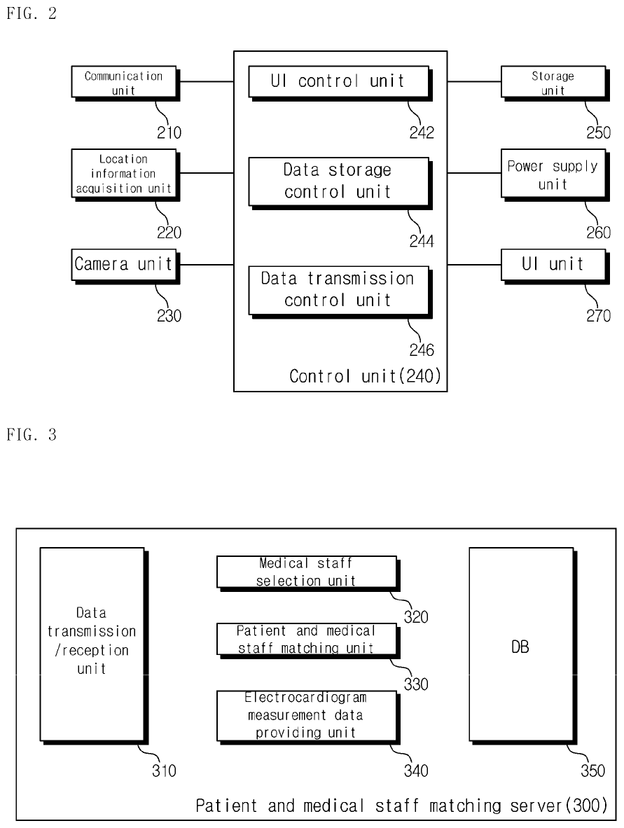

FIG. 2 is an exemplary block configuration diagram of a patient service app module 200 installed and executed in a patient's mobile communication terminal according to an embodiment of the present invention.

Referring to FIG. 2, the patient's mobile communication terminal comprises: a communication unit 210 including short-distance communication module for data transmission and reception with a wearable electrocardiogram measurement device 100 and a communication module for transmitting and receiving data with a patient and medical staff matching server 300 through a mobile communication network;

a location information acquisition unit 220 for acquiring the current location information of the terminal together with a GPS module;

a camera unit 230 for acquiring an image of a subject;

a storage unit 250 in which control program data for performing the overall control operation of the terminal and personal electrocardiogram measurement data transmitted from the wearable electrocardiogram measurement device 100, and program data constituting various service apps are stored;

a power supply unit 260 for supplying operating power of the terminal;

a user interface (UI) unit 270 that receives a command from a patient corresponding to a terminal user, displays the result of an electrocardiogram reading, and various display information on the display unit; and a control unit 240 for controlling the overall operation of the terminal. Meanwhile, the patient service app module stored in the storage unit 250 is executed by the control unit 240, and thereby receiving the electrocardiogram measurement data obtained from the wearable electrocardiogram measurement device 100, transmitting it to the patient and medical staff matching server 300 together with information on the request for electrocardiogram reading, receiving and displaying information on a list of medical staff responding to the request for electrocardiogram reading, and transmitting the medical staff selected by the patient as feedback.

As illustrated in FIG. 2, this patient service app module includes:

a UI control unit 242, which is configured to include an operation mode selection button for selecting an all-time measurement mode and a symptom expression measurement mode, a reading mode selection button for selecting a real-time reading and a full reading, and displaying medical staff list information on the display unit of the UI unit 270;

a data storage control unit 244 that divides the received patient's electrocardiogram measurement data into date, measurement time, and operation modes (all-time measurement mode, symptom expression measurement mode) and stores them in the storage unit 250; and

a data transmission control unit 246 that receives or transmits the patient's electrocardiogram measurement data between the wearable electrocardiogram measurement device 100 and the patient and medical staff matching server 300 depending on the selected operating mode or reading mode.

The data storage control unit 244, may select and store the electrocardiogram measurement data acquired for the previous n seconds (for example, 60 seconds) and the electrocardiogram measurements data acquired for the next m seconds (for example, 30 seconds) based on the input time when the symptom expression measurement mode selection is inputted. The selected and stored electrocardiogram measurement data can be transmitted together with real-time reading request information. (The above n and m are natural numbers.) For reference, the ‘symptom expression measurement mode’ is defined as a mode for measuring electrocardiogram data when cardiac-related symptoms (palpitation, dyspnea, and the like) are expressed in a patient.

Meanwhile, various embodiments described herein may be implemented in a recording medium readable by a computer or similar device using, for example, software, hardware, or a combination thereof.

According to the hardware implementation, the embodiments described herein may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and other electrical units for performing functions. In some cases, such embodiments may be implemented by a control unit 200.

According to the software implementation, embodiments such as a procedure or function may be implemented together with a separate software module for performing at least one function or operation. The software code may be implemented by a software application written in a suitable programming language. The software code may be stored in the storage unit 250 and executed by the control unit 200

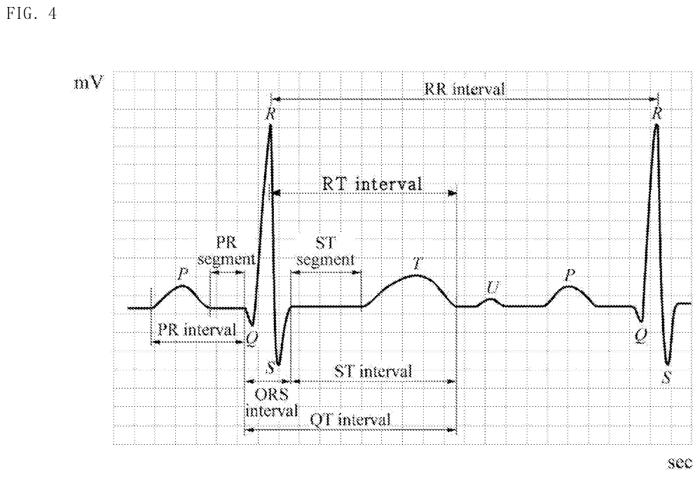

FIG. 3 illustrates a configuration diagram of a patient and medical staff matching server 300 according to an embodiment of the present invention, the patient and medical staff matching server 300 may comprise:

a data transmission/reception unit 310 for transmitting and receiving patient's electrocardiogram measurement data, medical staff information, the result of the readings with a mobile communication terminal 200 installed with a patient service app module;

a medical staff selection unit 320 that reads the patient's electrocardiogram measurement data using a deep learning trained artificial intelligence network model, and based on the result of the readings, selects some of the registered medical staff, and transmits a request for an electrocardiogram reading to some registered medical staff communication terminals 400; a patient and medical staff matching unit 330 that matches the selected medical staff according to the medical staff selection criteria and the patient who requested the electrocardiogram reading;

an electrocardiogram measurement data providing unit 340 that selects the patient's electrocardiogram measurement data provided from the patient or provides it to the selected or sorted medical staff communication terminal 400; and

a database (DB) 350 where medical staff information, electrocardiogram measurement data provided by the patient, and patient-specific information of the result of the reading are stored.

As an embodiment that can be implemented, the medical staff selection unit 320 may select only medical staff information close to the patient's current location information from among the registered medical staff information, regardless of real-time reading or full reading request, and transmit the request for electrocardiogram reading. In some cases, it may be possible to select medical staff by considering the distance between the patient and the medical staff only when full reading is requested.

As another possible embodiment, the medical staff selection unit 320 selects registered medical staff from patient's electrocardiogram measurement data according to the degree of the ratio of ventricular tachycardia (VT), superventricular tachycardia (SVT), atrial fibrillation (AF) to QRS, and rhythm detection, but may also select medical staff by selecting any one among the distance between the patient and the medical staff, the medical staff that can read in real-time, and the medical staff who responded first as the top priority selection criteria according to the degree of the ratio. For reference, the above VT and SVT are arrhythmias with abnormal rhythms, respectively, and tachycardia refers to a state in which a normal patient's heart rate becomes faster (more than 100 beats per minute). In the case of SVT, it refers to a phenomenon in which the beat suddenly accelerates, and when it repeats continuously, it is classified as an SVT rhythm. VT is a rapid pulse that occurs in the ventricles and is characterized by repeated ventricular premature beats occurring 3 or more times on the electrocardiogram, and AF, atrial fibrillation, is a disease that accounts for more than 70% of all arrhythmias, and is an arrhythmia that can progress to stroke and the like.

Meanwhile, the patient and medical staff matching unit 330 performs a role of providing the selected one or more medical staff list information to the patient service app module 200, receiving the feedback transmission of the medical staff information selected by a patient, matching the patient and the medical staff, and in accordance with this, registering the matching related information and the result of the readings obtained by the matched medical staff for each patient together with the electrocardiogram measurement data in a DB 35 and managing thereof.

Hereinafter, an operation in which the patient service app module 200 and a patient and medical staff matching server 300 including the above-described configurations interoperate to provide an electrocardiogram analysis matching support service will be described in detail.

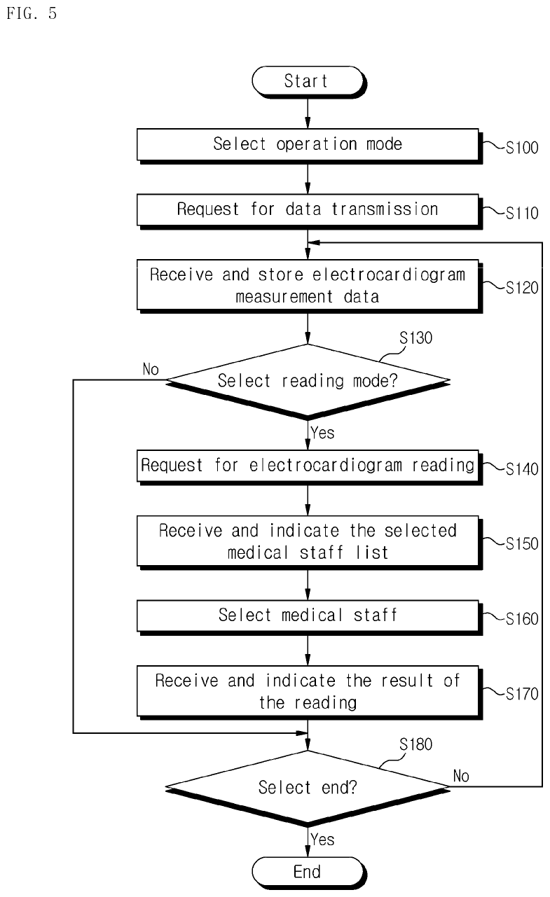

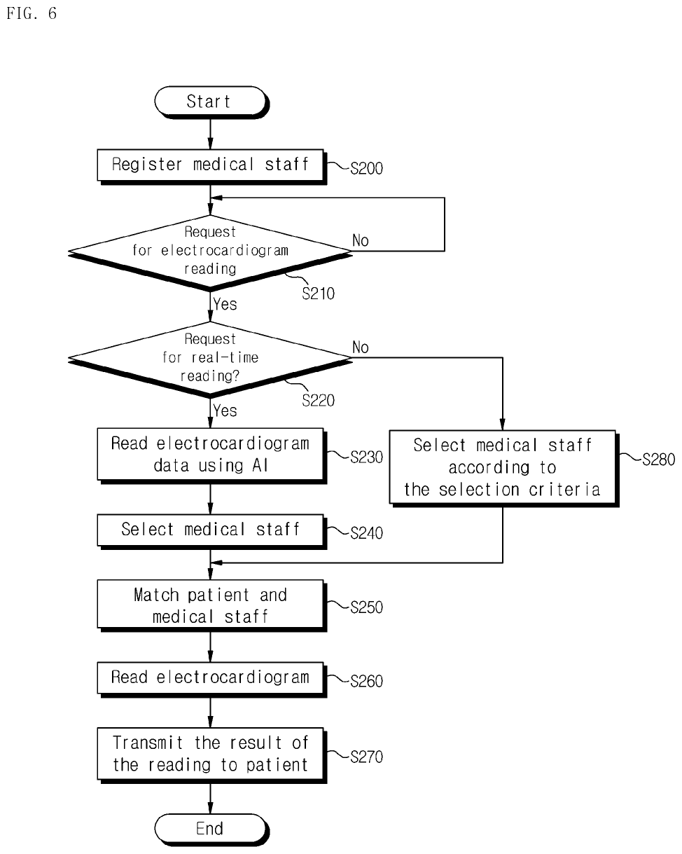

FIG. 4 is an exemplary view of an electrocardiogram waveform according to an embodiment of the present invention, FIG. 5 is an exemplary operation flow diagram of a patient service app 200 according to an embodiment of the present invention, and FIG. 6 is an exemplary operation flow diagram of a patient and medical staff matching server 300 according to an embodiment of the present invention.

First, a patient who wants to receive telemedicine by measuring his or her electrocardiogram attaches a wearable electrocardiogram measurement device 100 to a designated part of the body and operates it, and in addition, the patient executes the patient service app module 200 installed in his/her mobile communication terminal and selects an operation mode through the UI control unit 242 (step S100).

There are two types of operation modes: an all-time measurement mode for continuously measuring the electrocardiogram, such as a day or a week, and a symptom expression measurement mode. When an all-time measurement mode is selected by the patient while a Bluetooth connection is established between the wearable electrocardiogram measurement device 100 and the mobile communication terminal, the data transmission control unit 246 may request transmission of electrocardiogram measurement data toward the wearable electrocardiogram measurement device 100 (step S110).

In response to this data transmission request, the wearable electrocardiogram measurement device 100 transmits electrocardiogram measurement data obtained through a plurality of electrodes and signal-processed to the mobile communication terminal.

Accordingly, the data storage control unit 244 stores the patient's electrocardiogram measurement data received in real-time in the storage unit 250, but stores the date, measurement time, and operation mode separately (step S120).

When the measurement for a desired period is completed, the patient can apply for a full reading service. That is, if there is a read mode selection in step S130 and the read mode is full reading, the data transmission control unit 246 requests for electrocardiogram reading by transmitting the electrocardiogram measurement data acquired and stored in the all-time measurement mode to the patient and medical staff matching server 300 together with the full reading service request information (corresponding to information on the request for electrocardiogram reading) (step S140). When the full reading service is requested, current location information obtainable from the patient's mobile communication terminal may be further included and transmitted in order to satisfy the medical staff selection criteria.

After the request for electrocardiogram reading is made, as the medical staff is selected by the patient and medical staff matching server 300, the data transmission control unit 246 receives the medical staff list information selected from the patient and medical staff matching server 300, and displays it on the display unit constituting the UI unit 270 (step S150).

The medical staff list information includes the location, profile, and patient evaluation information of each selected medical staff. Accordingly, when the patient selects a medical staff suitable for his/her preference (step S160), the data transmission control unit 246 transmits the medical staff information selected by the patient as feedback.

In this way, when a medical staff is selected by a patient, the patient and medical staff matching server 300 matches the patient and the selected medical staff so that the selected medical staff receives and reads the patient's electrocardiogram measurement data. This patient-medical staff matching process will be described in detail with reference to FIG. 6.

The result of the reading conducted by the medical staff selected by the patient is transmitted to the patient service app module 200 through the patient and medical staff matching server 300 and displayed (step S170), so that the patient can receive a result of the reading of his/her electrocardiogram measurement data acquired in the all-time measurement mode. Accordingly, the patient can freely select an attending physician and receive an electrocardiogram diagnosis regardless of distance and time through the electrocardiogram analysis matching support service system according to an embodiment of the present invention.

In the above embodiment, the all-time measurement mode has been described, but when a symptom occurs while performing the all-time measurement mode, the patient may select the symptom expression measurement mode in the operation mode. Of course, the symptom expression measurement mode can be selected directly without performing the all-time measurement mode.

If the symptom expression measurement mode is selected by the patient, the data storage control unit 244 selects and stores the electrocardiogram measurement data acquired for the previous 60 seconds and the electrocardiogram measurements data acquired for the next 30 seconds based on the input time when the symptom expression measurement mode selection is inputted (step S120).

Because symptom expression has occurred, the patient can choose to take a real-time reading. That is, if there is a read mode selection in step S130 and the read mode is real-time reading, the data transmission control unit 246 requests for an electrocardiogram reading by transmitting the electrocardiogram measurement data selected and stored in the symptom expression measurement mode to the patient and medical staff matching server 300 together with information on the request for real-time reading service (corresponding to information on the request for electrocardiogram reading) (step S140).

After a request for real-time electrocardiogram reading is made, as the medical staff is selected by the patient and medical staff matching server 300, the data transmission control unit 246 receives the medical staff list information selected from the patient and medical staff matching server 300 and displays it on the display unit constituting the UI unit 270 (step S150). Since the process after step S150 is the same as that described above, it will be omitted below. However, in the case of real-time reading, there may be cases in which urgency is required, so depending on the implementation method, the reading may be performed by matching the patient and the medical staff immediately without the selection process of the medical staff.

Since real-time reading is performed for the electrocardiogram measurement data obtained in the symptom expression measurement mode described above, there is an advantage in that an individual's electrocardiogram can be analyzed and provided in real-time in a timely manner.

The operation of the patient and medical staff matching server 300 is further described hereinafter with reference to FIG. 6.

First, medical staffs who wish to subscribe to the electrocardiogram interpretation matching support service register medical information including their profile, location, and field of study in the system (step S200). It is preferable that information indicating a service field, such as a medical staff capable of providing a real-time reading service and a medical staff capable of providing a full reading service, is also registered in the registered medical staff information.

When there is a request for electrocardiogram reading from the patient service app module 200 through the data transmission/reception unit 310 (step S210), the medical staff selection unit 320 determines whether the electrocardiogram read request is a real-time reading request. Whether the request for electrocardiogram reading is a request for a real-time reading or a request for a full reading will not be explained further here since it has been described with reference to FIG. 5 above.

If it is a request for a real-time reading, the medical staff selection unit 320 reads the patient's electrocardiogram measurement data transmitted along with information on the request for electrocardiogram reading using the deep learning-learned artificial intelligence (AI) network model (step S230), and selects some of the registered medical staff according to the result of the reading (step S240), and transmits the request for electrocardiogram reading. For example, the medical staff selection unit 320 selects the medical staff among medical staff who can provide real-time reading service, the medical staff currently on standby, and the medical staff with the best patient evaluation as the top priority selection criteria in cases that require faster reading, such as Tachycardia, Bradycardia, Atria Flutter, and the like as a result of the reading the electrocardiogram measurement data using the AI network model (step S240)

As another example, the medical staff selection unit 320 reads the electrocardiogram measurement data using the AI network model, and selects the medical staff with reference to the ratio of normal and abnormal bits. For example, among the number of QRS complexes (R-peak), VT, and SVT, if VT and SVT to the number of QRS complexes exceeds 2% (number of QRS complexes: V, number of QRS complexes: S), the medical staff is selected among the available medical staff who can provide real-time reading service, and the medical staffs who are currently marked as standby state as the top priority selection criteria (step S240).

As a result of the reading the electrocardiogram measurement data using the AI network model, among the number of QRS complexes (R-peak), VT, and SVT, if VT and SVT to the number of QRS complexes does not exceed 2% (number of QRS complexes: V, number of QRS complexes: S), the medical staff who respond first to requests for reading is selected as the top priority selection criteria (step S240). Even if a real-time reading is requested the medical staff may be selected by simply selecting the minimum distance between the patient and the medical staff as the top priority selection criteria in consideration of the patient's prior request and age. In some cases, when the AF Burden exceeds 2%, the medical staff selection unit 320 may select the medical staff by increasing the priority.

For reference, the VT and SVT are abnormal rhythms, that is, arrhythmias as previously summarized, and abnormal beats corresponding to each are V (or PVC-premature ventricular contraction) and S (or PAC-prematureatrial contraction or SVPB-supraventricular or ectopic beat). V gathers in a specific pattern to form a VT rhythm, and S gathers to form an SVT rhythm. The above numerical value 2% is a threshold value preset to classify the severity, and the severity can be calculated as a ratio of abnormality to all bits (normal and abnormal). In addition, the above value of 2% may vary depending on the system, internal policies, expert consultations, and experimental results.

As described above, when the medical staff is selected by the medical staff selection unit 320, the patient and medical staff matching unit 330 requests an electrocardiogram reading to the medical staff communication terminal 400, or after the selection of medical staff is made by propagating information on the selected medical staff list to the patient service app module 200 that requested reading, a request for electrocardiogram reading is sent to the corresponding medical staff.

When the selected medical staff responds to the request for electrocardiogram reading, the electrocardiogram measurement data providing unit 340 provides the patient's electrocardiogram measurement data requested for the electrocardiogram reading to the selected medical staff communication terminal 400, so that the corresponding medical staff can leave the result of the electrocardiogram reading (step S260).

This result of the electrocardiogram reading is registered in the DB 350 of the matching server 350 and transmitted to the patient service app module 200 (step S270), so that the patient who requested electrocardiogram reading can receive the result of the reading for the electrocardiogram measurement data obtained before and after symptom expression.

Meanwhile, if the request is for full reading request not real-time reading in step S220, the medical staff selection unit 320 selects the medical staff according to the medical staff selection criteria according to the request for full reading (step S280). The medical staff selection criteria here are based on internal standards: the medical staff located closest to the patient may be the highest selection criterion; the medical staff who respond in advance may be the highest priority selection criterion; the medical staff who responds first among the medical staff located within a certain distance from the patient can be selected as the highest priority selection criterion; and a medical staff with the best patient evaluation may be the highest priority selection criterion.

A series of processes of matching the medical staff and the patient selected according to the highest priority screening criteria, making requests for reading, and transmitting the result to the patient side are the same as the steps S250 to S270 described above, and thus will be omitted hereinafter. As described above, since the electrocardiogram analysis matching support service system according to the embodiment of the present invention can match the best medical staff in a timely manner by applying different medical staff selection criteria according to the reading mode selected by the patient, there are advantages in that a timely and real-time analysis of an individual's electrocardiogram requested to be read from a remote location can be provided, and the patient can freely select an attending physician regardless of distance and time to receive an electrocardiogram diagnosis. In addition, since the patient registers his/her electrocardiogram measurement data in his/her communication terminal or the patient and medical staff matching server 300 and uses it whenever necessary, it is possible to minimize the payment for receiving medical data, and medical staffs can also enjoy the convenience of providing telemedicine services to patients who need them by accessing the online system whenever necessary.

In the above, the present invention has been described by specific matters such as specific components and limited embodiments and drawings, but these are only provided to help a more general understanding of the present invention, and the present invention is not limited to the above examples, and those of ordinary skill in the art to which the present invention pertains can devise various modifications and variations from these descriptions. Therefore, the spirit of the present invention should not be limited to the above-described embodiments, and not only the claims to be described later but also all equivalent or equivalent variations to these claims shall fall within the scope of the spirit of the present invention.

1-12. (canceled)

13. An electrocardiogram analysis matching support service system, characterized in that it comprises:
a patient service app module, installed and executed on a mobile communication terminal of a patient, that transmits an electrocardiogram measurement data received from a wearable electrocardiogram measurement device to a patient and medical staff matching server to request for a reading, and receives and displays the result of the reading; and
a patient and medical staff matching server that reads the electrocardiogram measurement data transmitted from the patient service app module using a deep learning artificial intelligence network model, selects a pre-registered medical staff according to the result of the reading, and supports to read the electrocardiogram measurement data.

14. The electrocardiogram analysis matching support service system according to claim 13, wherein
the patient service app module is characterized by comprising:
a UI control unit, which is configured to include an operation mode selection button to select a continuous measurement mode and a symptom expression measurement mode, a reading mode selection button to select a real-time reading and a full reading, and displaying medical staff list information;
a data storage control unit that divides the received patient's electrocardiogram measurement data into a date, measurement time, and operation mode and stores it in the patient's mobile communication terminal storage unit; and
a data transmission control unit for receiving or transmitting control of the patient's electrocardiogram measurement data between the wearable electrocardiogram device and the patient and medical staff matching server according to the selected operation mode or reading mode.

15. The electrocardiogram analysis matching support service system according to claim 14,
wherein the data storage control unit is characterized by selecting and storing the patient's electrocardiogram measurement data acquired for the previous n seconds and the patient's electrocardiogram measurement data acquired for the next m seconds based on the input time when the symptom expression measurement mode selection input is inputted, and
wherein the n and in are natural numbers.

16. The electrocardiogram analysis matching support service system according to claim 13,
wherein the patient service app module is characterized by transmitting the current location information obtainable from the patient's mobile communication terminal together with the information of the request for electrocardiogram reading.

17. The electrocardiogram analysis matching support service system according to claim 16,
wherein the patient and medical staff matching server is characterized by selecting only medical staff information close to the patient's current location information from among the registered medical staff information, and propagating the request for electrocardiogram reading.

18. The electrocardiogram analysis matching support service system according to claim 13,
wherein the patient and medical staff matching server is characterized by further including a medical staff selection unit that reads patient's electrocardiogram measurement data using deep learning trained artificial intelligence network model, selects some of the registered medical staff according to the result of the reading, and transmits the requests for electrocardiogram reading.

19. The electrocardiogram analysis matching support service system according to claim 14,
wherein the patient and medical staff matching server is characterized by further including a medical staff selection unit that reads patient's electrocardiogram measurement data using deep learning trained artificial intelligence network model, selects some of the registered medical staff according to the result of the reading, and transmits the requests for electrocardiogram reading.

20. The electrocardiogram analysis matching support service system according to claim 15,
wherein the patient and medical staff matching server is characterized by further including a medical staff selection unit that reads patient's electrocardiogram measurement data using deep learning trained artificial intelligence network model, selects some of the registered medical staff according to the result of the reading, and transmits the requests for electrocardiogram reading.

21. N The electrocardiogram analysis matching support service system according to claim 16,
wherein the patient and medical staff matching server is characterized by further including a medical staff selection unit that reads patient's electrocardiogram measurement data using deep learning trained artificial intelligence network model, selects some of the registered medical staff according to the result of the reading, and transmits the requests for electrocardiogram reading.

22. The electrocardiogram analysis matching support service system according to claim 18,
wherein the medical staff selection unit is characterized by selecting registered medical staff from patient's electrocardiogram measurement data according to the degree of anyone among the ratio of VT, SVT, and AF to QRS complex number, and by selecting medical staff by selecting anyone among the distance between the patient and the medical staff, the medical staff that can read in real-time, and the medical staff who responded first as the top priority selection criteria according to the degree of the ratio.

23. The electrocardiogram analysis matching support service system according to claim 19,
wherein the medical staff selection unit is characterized by selecting registered medical staff from patient's electrocardiogram measurement data according to the degree of anyone among the ratio of VT, SVT, and AF to QRS complex number, and by selecting medical staff by selecting anyone among the distance between the patient and the medical staff, the medical staff that can read in real-time, and the medical staff who responded first as the top priority selection criteria according to the degree of the ratio.

24. The electrocardiogram analysis matching support service system according to claim 20,
wherein the medical staff selection unit is characterized by selecting registered medical staff from patient's electrocardiogram measurement data according to the degree of anyone among the ratio of VT, SVT, and AF to QRS complex number, and by selecting medical staff by selecting anyone among the distance between the patient and the medical staff, the medical staff that can read in real-time, and the medical staff who responded first as the top priority selection criteria according to the degree of the ratio.

25. The electrocardiogram analysis matching support service system according to claim 21,
wherein the medical staff selection unit is characterized by selecting registered medical staff from patient's electrocardiogram measurement data according to the degree of anyone among the ratio of VT, SVT, and AF to QRS complex number, and by selecting medical staff by selecting anyone among the distance between the patient and the medical staff, the medical staff that can read in real-time, and the medical staff who responded first as the top priority selection criteria according to the degree of the ratio.

26. An electrocardiogram analysis matching support service system characterized by including:
a patient service app module installed and executed in a patient's mobile communication terminal, transmitting the electrocardiogram measurement data received from a wearable electrocardiogram measurement device to request a reading, and receiving and displaying the result of the reading; and
a patient and medical staff matching server that reads the electrocardiogram measurement data transmitted from the patient service app module using a deep learning trained artificial intelligence network model, selects a pre-registered medical staff according to the result of the reading, and supports reading the electrocardiogram measurement data

27. The electrocardiogram analysis matching support service system according to claim 26,
Wherein the patient service app module is characterized by selecting, storing, and transmitting the patient's electrocardiogram measurement data acquired for the previous n seconds and the patient's electrocardiogram measurement data acquired for the next m seconds based on the input time when the symptom expression measurement mode selection input is inputted, and
wherein the n and m are natural numbers.

28. The electrocardiogram analysis matching support service system according to claim 26,
wherein the patient service app module is characterized by transmitting the current location information obtainable from the patient's mobile communication terminal together with the information of the request for electrocardiogram reading.

29. The electrocardiogram analysis matching support service system according to claim 26,
wherein the patient and medical staff matching server is characterized by including a medical staff selection unit that reads patient's electrocardiogram measurement data using deep learning trained artificial intelligence network model, selects some of the registered medical staff according to the result of the reading, and requests for electrocardiogram reading.

30. The electrocardiogram analysis matching support service system according to claim 29,
wherein the medical staff selection unit is characterized by selecting registered medical staff from patient's electrocardiogram measurement data according to the degree of the ratio of anyone among VT, SVT, and AF to QRS complex number, and by selecting medical staff by selecting anyone among the distance between the patient and the medical staff, the medical staff that can read in real-time, and the medical staff who responded first as the top priority selection criteria according to the degree of the ratio.
